(19) United States
(12) Patent Application Publication
(10) Pub. No.: US 2022/0161010 A1
(43) Pub. Date: May 26, 2022

(54) DRUG DELIVERY AND ADMINISTRATION DEVICE

(71) Applicant: CALLA LILY PERSONAL CARE LTD, London (GB)

(72) Inventors: Yu Sing (Alex) HOOI, London (GB); Vinh-Thang VO-TA, London (GB)

(21) Appl. No.: 17/601,215

(22) PCT Filed: Apr. 3, 2020
(86) PCT No.: PCT/GB2020/050891

§ 371 Date: Oct. 4, 2021

(30) Foreign Application Priority Data
Apr. 5, 2019 (GB) 1904856.0

(51) Int. Cl. A61M 31/00

(52) U.S. Cl. CPC A61M 31/002; A61M 2202/0007; A61M 31/007

(57) ABSTRACT

A device for the delivery and/or application of a therapeutic composition in a delivery form into the rectal and/or vaginal cavity, the device includes an externally wearable pad; a non-absorbent platform for receiving the delivery form of therapeutic composition; and a sheath joining the platform to the pad such that a user's finger can be received in the sheath.

The present invention relates to a device for the delivery and/or administration of a therapeutic composition in the rectal or vaginal cavity.

The delivery of drugs through the vaginal or rectal cavity is known to be advantageous in that both local and systemic administration of the drug can be achieved and provides an alternative to the traditional delivery routes. In some instances, oral delivery is not recommended because of gastro-intestinal side effects or decrease in bioavailability or simply because the patient is uncooperative or cannot swallow the medication. Parenteral drugs (e.g. intravenous injections) might not be suitable, as they must usually be administered by trained practitioners.

Vaginal and rectal drugs (such as creams) can be applied with a gloved finger. However, when the finger is inserted into the vaginal or rectal cavity, a portion of the drug that will be retained by the vaginal or rectal walls will not be applied to the targeted area as the finger advances through the cavity. It is therefore difficult to administer an accurate amount of drug to the targeted area.

Drugs can also be delivered by inserting suppositories or by injecting the drug(s) using a syringe or a spray. However, due to their physical function, both vaginal and rectal cavities have a tendency to reject foreign bodies. The drugs might reach the targeted area but are not retained within the cavity so as to allow sufficient contact and absorption to be achieved. This is particularly the case where the suppository must dissolve to allow drug penetration and absorption into the cavity walls. Elastomeric vaginal rings have been developed to retain the leaking drug, but these devices are often uncomfortable for the patient.

A number of medicated tampons and sponges are known for delivery of drugs to the vaginal or rectal cavity. These devices are usually inserted using an applicator or inserter, which is uncomfortable and it is often difficult to properly position the tampon.

European patent publication EP 3 027 155 is directed to a drug delivery device comprising a medicated tampon joined to a sanitary pad by a sheath. The user can insert his/her finger in the sheath in order to push the tampon into the vaginal or rectal cavity, accurately and atraumatically. However, there may be medications which would require substantial modifications and costs to be incorporated into a tampon, or simply cannot be incorporated into a tampon at all.

It is an object of the invention to at least alleviate the above-mentioned disadvantages, or to provide an alternative to existing products.

According to a first aspect of the invention, there is provided a device for the delivery and/or application of a therapeutic composition in a delivery form into the rectal and/or vaginal cavity, the device comprising an externally wearable pad; a non-absorbent platform for receiving the delivery form of therapeutic composition; and a sheath joining the platform to the pad such that a user's finger can be received in the sheath.

In the context of the present invention, the delivery form can be any commercially available delivery form including but not limited to a suppository, pessary, capsule, tablet, liquid, solution, suspension, lotion, dispersion, cream, gel, powder and granules. The present invention is not directed to the delivery and administration of therapeutic compositions delivered in the form of gas.

Known rectal or vaginal delivery devices require the absorption or combination of the drug with the delivery device. Although this facilitates the control of the release of the drug, there is still the risk that the entire dosage will be retained by the delivery device and not be delivered to the target area. The present invention enables the entire therapeutic composition (i.e. in its delivery form) to be delivered to the target area, in that there is no absorption involved. It could be said that the present device acts as an applicator, rather than a delivery device, in that there is no interaction between the device and the therapeutic composition. It could also be said that the therapeutic composition is supported by the platform.

Preferably, the sheath comprises said platform. The sheath comprises a first end which is joined to the pad, and a second end which comprises the platform, so that the platform can be pushed into the rectal or vaginal cavity by the tip of the user's finger.

In a preferred embodiment, the therapeutic composition is a delivery form with a shape, and the platform comprises a receiving portion, the shape of which corresponds partially to the shape of the delivery form of the therapeutic composition.

Within the context of the present invention, we will refer to a shape of the delivery form of a therapeutic composition where the delivery form consists of one component, e.g. one suppository, one pessary, one capsule, one tablet; but not a liquid, solution, suspension, lotion, dispersion, cream, gel, powder and granules, which in their delivery form have no specific shape.

The shape of the delivery form may be spherical, oval, cylindrical, a lozenge or any other commercially available shape. The shape of the receiving portion preferably corresponds partially to the shape of the delivery form so that the therapeutic composition can fit snuggly in the receiving portion, yet be released at the target area. Preferably, the shape of the receiving portion corresponds to up to half the shape of the delivery form of the therapeutic composition to enable easy release at the target site.

If the shape of the delivery form is spherical or an oval capsule, then the platform may comprise a semi-spherical or egg cup shaped receiving portion. If the shape of the delivery form is a lozenge, the platform may comprise a tapered receiving portion.

The receiving portion may be integrally formed in the sheath. For example, the sheath may comprise a receiving portion (as a pocket or recess) formed in the same material as the sheath. Alternatively, the receiving portion may comprise an additional or alternative material, which connected to the sheath.

This specific configuration of the receiving portion is advantageous in that it improves the delivery of the therapeutic composition. The sheath is such that a user's finger can be received therein. This can be achieved by using a sheath of sufficient diameter, or a sheath material which can expand to accommodate a finger. Consequently, the diameter of the sheath is likely to be wider than the dimensions of the delivery form so that the tablet, capsule, suppository or other, will be loose if not retained by a shaped receiving portion and therefore the user will experience difficulties in pushing the therapeutic composition into the rectal or vaginal cavity.

Preferably, the platform comprises a receiving portion which is adapted and configured to receive a predetermined amount of therapeutic composition.

In a preferred embodiment, the receiving portion may be a receptacle, such as a cup or bowl. Preferably, the receiving portion has a rounded base so that to prevent the therapeutic composition to be trapped in corners and undelivered.

This is particularly advantageous when the delivery form of the therapeutic composition is not definite, for example where the therapeutic composition is in a delivery form in which it is difficult to quantify, e.g. if the delivery form is a liquid, solution, suspension, lotion, dispersion, cream, gel, powder and granules. If the receiving portion is adapted and configured to receive a predetermined amount of therapeutic composition, the user can simply fill up the receiving portion with the exact amount of therapeutic composition required for the treatment.

In another embodiment, the receiving portion may comprise a visual graduation or marker such that the user can adjust the amount of therapeutic composition required for the treatment. The amount required may depend for example on the condition to be treated, on the severity of the condition, on the patient's characteristics such as age, weight and sex.

Preferably, the platform comprises an area of relatively greater rigidity. The platform may comprise or be made of a material which is more rigid than the material of the sheath. An additional or alternative material may be used to increase the rigidity of the platform and/or receiving section. For example, the area of relatively greater rigidity can have a multilayer structure or be made of a different, more rigid material.

This would enable the platform and/or receiving section to maintain its shape as it is advanced into the rectal or vaginal cavity. If the shape of the receiving portion collapsed, then a liquid therapeutic composition contained therein could spill or spread into the sheath, thereby rendering the delivery to the target site more difficult and/or resulting in an inaccurate amount of the therapeutic composition to be delivered to the target site. If the therapeutic composition is in the shape of a single solid form (e.g. tablet, capsule, suppository, bougie or pessary), the composition may become accidentally dislodged from the receiving platform before reaching the target area.

The area of greater rigidity may also be used to increase the robustness of the platform so as to allow a removal string to be attached thereto.

Preferably, the receiving portion is collapsible and/or reversible. In a preferred embodiment, the receiving portion is rigid enough to maintain its shape during the insertion step, i.e. when the therapeutic composition is pushed through the rectal or vaginal cavity towards the target area, but flexible enough to be collapsed and to release the therapeutic composition at the target area. It is preferred that the device according to the present invention does not comprise any fully rigid components so as to avoid injury and discomfort to the user during the insertion and removal processes.

In a preferred embodiment, the platform comprises means for releasing the therapeutic composition.

In the simplest embodiment, the platform is reversible so that the therapeutic composition is pushed out of the receiving portion. For example, the platform comprises or is a cup- or bowl-shaped receptacle which can be reversed out. Once reversed, the platform can be used to apply the therapeutic composition onto the target area for example in the case of a cream, lotion, liquid, granule and the like, so that the composition penetrates the cavity walls.

The platform may be collapsible or the platform may comprise collapsible side walls so that the therapeutic composition is released from the receiving section. The platform may comprise one or more strings configured and arranged to be pulled towards the cavity orifice and collapse the side walls. For example, the platform walls may comprise a concertina mechanism.

In a preferred embodiment, the receiving portion may be closable so as to contain the therapeutic composition for example during storage, transport, handling and insertion of the device. The sheath may be releasably closed, for example by ultrasonic welding, impulse heat or heat welding. These techniques are particularly advantageous in that the strength of the seal can be adjusted so that the closure is strong enough to contain the therapeutic composition before use, but can also be opened by pushing the sheath into the patient's cavity. The receiving portion is pushed to the target area and the user pushes the distal end of the sheath further so as to open the seal and release the therapeutic composition from the receiving portion.

Preferably, the length of the sheath is substantially the same as the distance between the rectal or vaginal orifice a target area in the rectal or vaginal cavity. If the sheath comprises a releasable closure, then the length of the sheath between the pad and the closure is preferably substantially the same as the distance between the rectal or vaginal orifice a target area in the rectal or vaginal cavity.

In a preferred embodiment, the device further comprises a removal string. Preferably, the device comprises a string for releasing the therapeutic composition from the receiving portion and/or for removing the device from the rectal or vaginal cavity. Preferably, the string is connected to the platform, or to the end of the sheath comprising the platform. In another embodiment, the string comprises a loop or forms a loop, so that it can be easily grabbed by the user. The loop may be connected to a plurality of points on or adjacent the platform so as to uniformly manipulate and/or pull the device, platform or receiving portion.

In a preferred embodiment, the pad comprises an absorbent layer facing, in use, the rectal or vaginal cavity and an impermeable layer facing, in use, away from the rectal or vaginal cavity.

The externally worn pad may comprise an absorbent layer to absorb any excess therapeutic composition or bodily fluid leaking from the cavity. This is particularly advantageous in the case of the application of a delivery form such as a liquid, cream or gel which is more likely to leak out of the cavity. The externally worn pad may comprise an impermeable layer to prevent the leaking fluid from staining the user's underwear. It is preferred that the pad is positioned outside the vaginal orifice as an interlabial pad or between the user's buttocks outside the rectal orifice. In a preferred embodiment, the pad comprises one or more lines of weakness to facilitate retention of the pad between the labia or the user's buttocks. The line of weakness also allows for a hygienic disposal of the drug delivery device, because the pad can be folded about the line of weakness, thereby closing the sheath so that the user does not touch the soiled surface of the pad or the sheath.

According to a second aspect of the present invention, there is provided a kit comprising a therapeutic composition in a delivery form and a device according to any one of claims 1 to 9 for delivering and/or applying said delivery form of the therapeutic composition to a target area in the rectal or vaginal cavity.

Preferably, the therapeutic composition comprises an anti-fungal agent, an anti-microbial agent, an anti-inflammatory agent, a pain killer, a steroidal (NSAID) compound, a non-steroidal compound, and/or a hormone.

According to a further aspect of the present invention, the device may be used for the delivery and/or application of a therapeutic composition for the treatment of menorrhagia.

According to a further aspect of the present invention, the device may be used for the delivery and/or application of a therapeutic composition for fertility treatment.

The invention will be further described with reference to the drawings and figures, in which

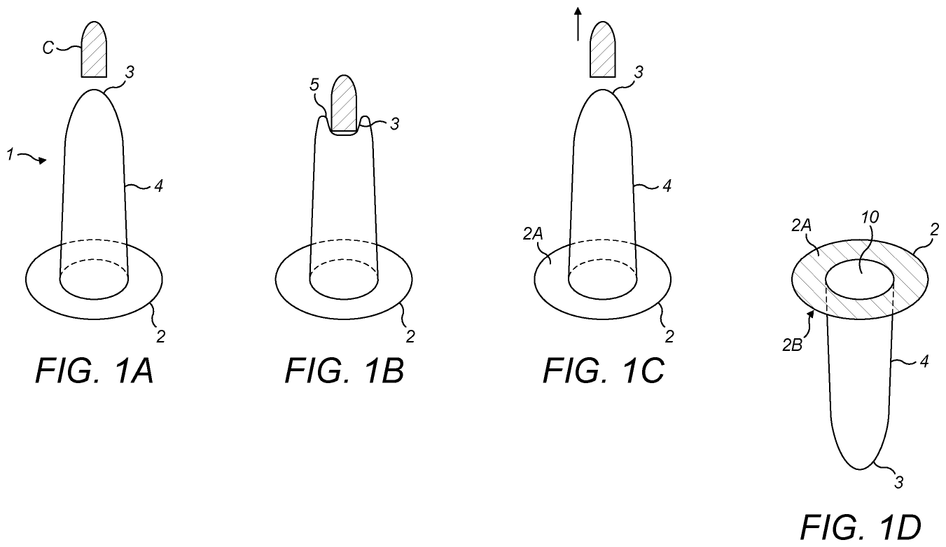

FIGS. 1A to 1D are schematic representations of a first device according to the present invention;

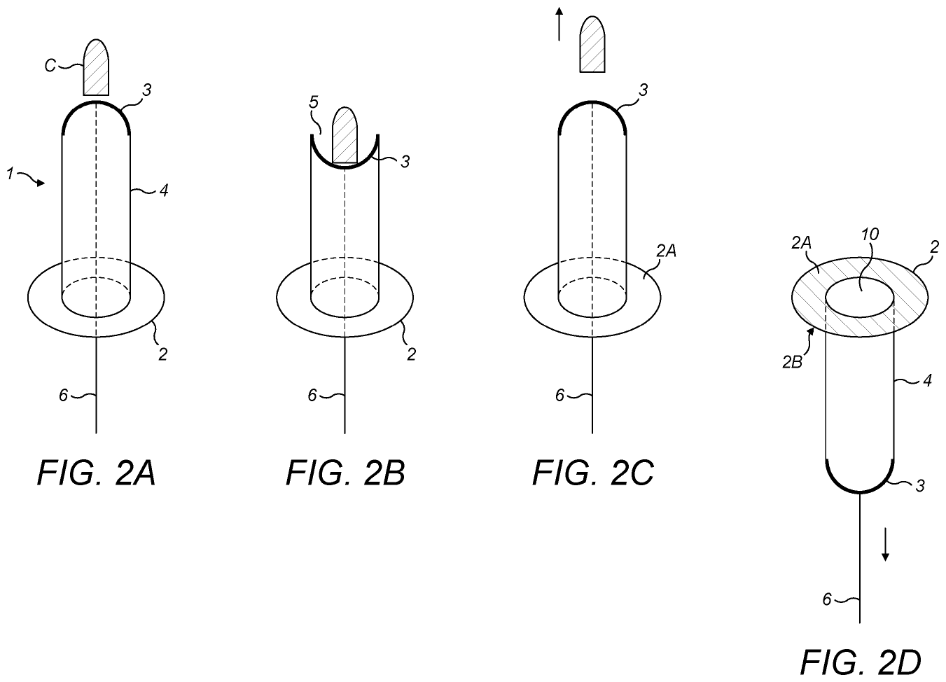

FIGS. 2A to 2D are schematic representations of a second device according to the present invention;

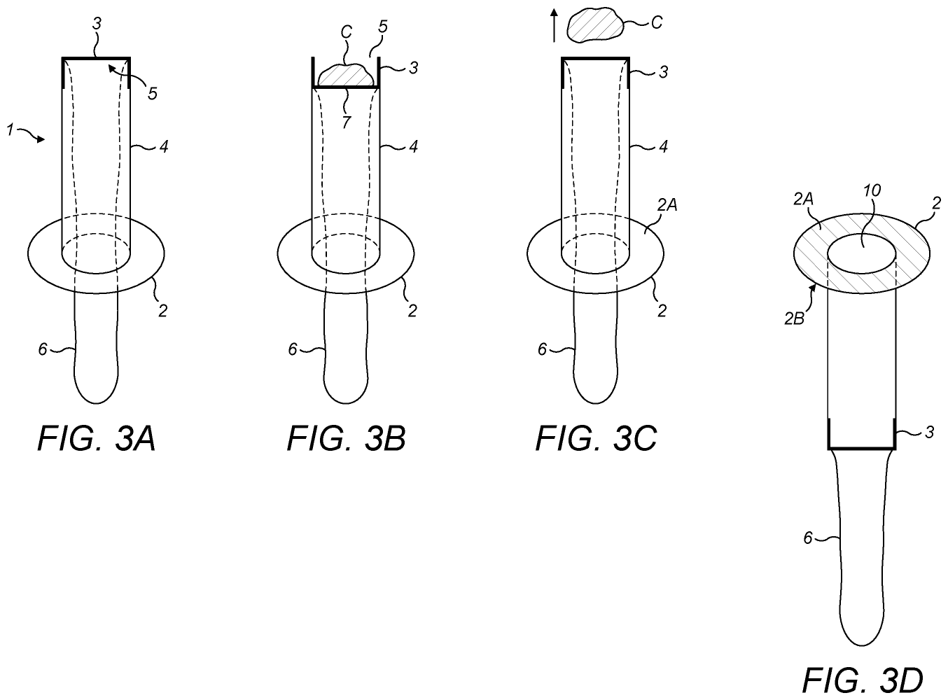

FIGS. 3A to 3D are schematic representations of a third device according to the present invention;

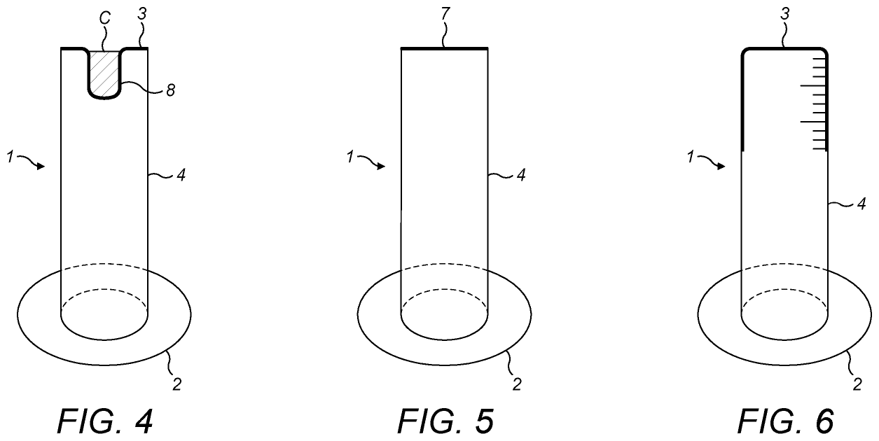

FIG. 4 is a schematic representation of a fourth device according to the present invention;

FIG. 5 is a schematic representation of a fifth device according to the present invention;

FIG. 6 is a schematic representation of a sixth device according to the present invention;

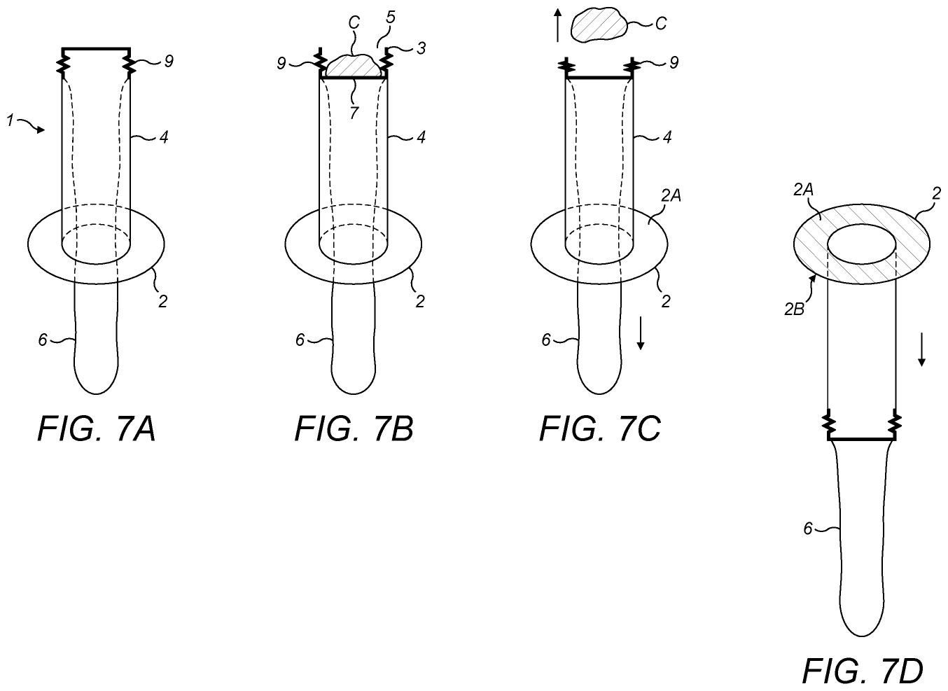

FIGS. 7A to 7D are schematic representations of a seventh device according to the present invention;

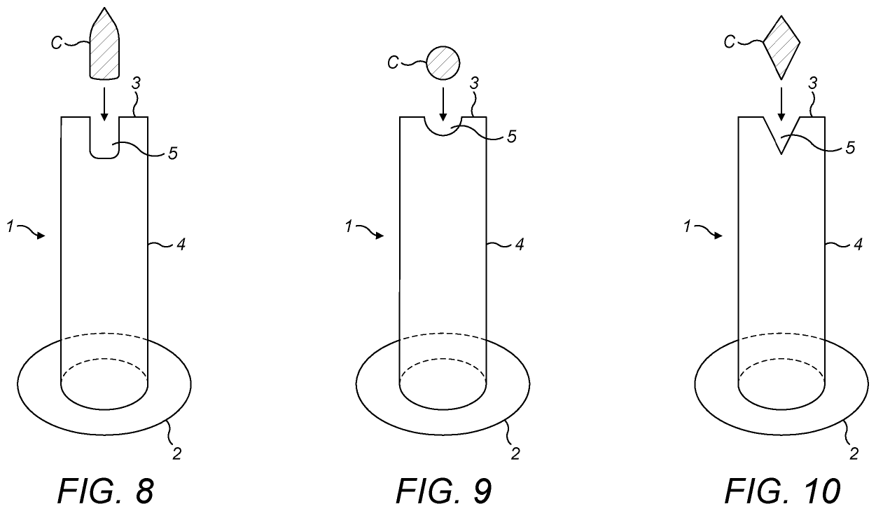

FIG. 8 is a schematic representation of an eighth device according to the present invention;

FIG. 9 is a schematic representation of a ninth device according to the present invention;

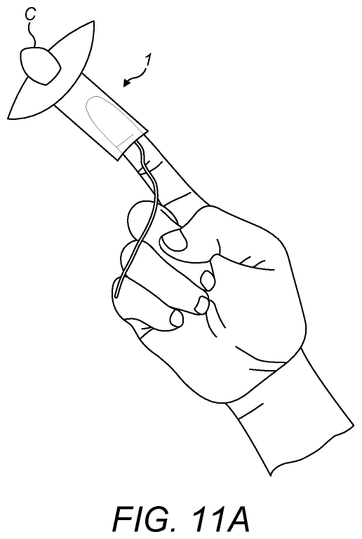

FIG. 10 is a schematic representation of a tenth device according to the present invention;

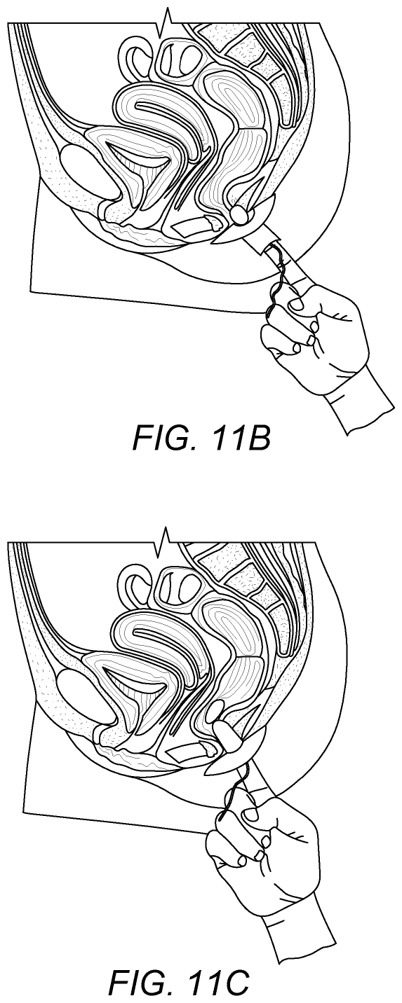

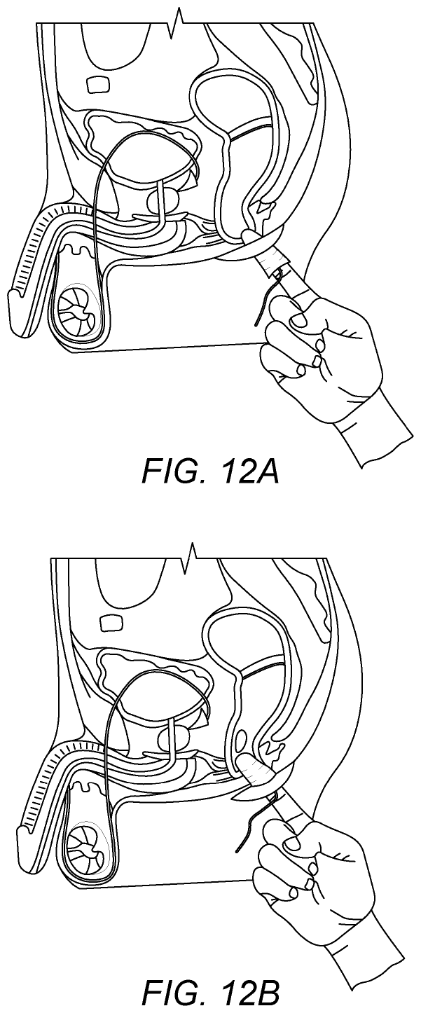

FIGS. 11A to 11C are schematic representations illustrating the delivery into the rectal cavity of a female patient of a therapeutic composition using a device according to the present invention; and

FIGS. 12A and 12B are schematic representations illustrating the delivery into the rectal cavity of a male patient of a therapeutic composition using a device according to the present invention.

Referring to FIG. 1, there is illustrated a device 1 for the delivery and/or application of a therapeutic composition C in a delivery form into the rectal and/or vaginal cavity, the device 1 comprising an externally wearable pad 2; a non-absorbent platform 3 for receiving the delivery form of therapeutic composition C; and a sheath 4 joining the platform 3 to the pad 2 such that a user's finger can be received in the sheath 4.

The non-absorbent platform 3 is located at the distal end of the sheath 4. Within the context of the present invention, the distal end is, in use, the end closest to the target area in the vaginal or rectal cavity and the proximal end is the end of the sheath 4 closest to the cavity orifice.

The primary function of the platform 3 is to provide a supporting surface for the therapeutic composition C and for the user to push said therapeutic composition C along the vaginal or rectal cavity. If the user was using a latex glove without a platform 3, the therapeutic composition C would move away from the user's fingertip and efficient and accurate delivery is not possible. The platform 3 also enable the user to maintain the therapeutic composition C at the target area in the vaginal or rectal cavity, and can act as an internal barrier to prevent the therapeutic composition C from becoming dislodged or leaking out of the cavity.

Preferably, the platform 3 comprises a material which is more rigid that the material of the sheath 4. The platform 3 may be made of or from a monolayer or a multilayer sheet structure. The material of the platform 3 may be the same material as that of the sheath 4 but which a thickness greater than the thickness of the material of the sheath 4 to impart relative rigidity. The platform 3 may comprise a multilayer structure comprising a structural layer to impart relative rigidity. A platform 3 of comparatively greater structural rigidity also facilitates the attachment of a string or cord 6.

The platform 3 comprises a receiving portion 5 or is capable of forming a receiving portion 5. The platform 3 may be arranged in a first configuration in which there is no receiving portion 5 (e.g. when it is reversed out in the delivery configuration at the target area in the cavity and/or in the retrieval configuration) and in a second configuration in which there is a receiving portion 5 (e.g. in the insertion configuration, when the therapeutic composition C is pushed along the vaginal or rectal cavity). In a preferred embodiment, the receiving portion 5 may be arranged in a receiving configuration in which it can contain a therapeutic composition C and in a delivery configuration in which the therapeutic composition C is ejected from the receiving portion 5.

The platform 3 and the receiving portion 5 can be made of the same non-absorbent material or different absorbent materials. In a preferred embodiment, the receiving portion 5 is integrally formed in and/or with the platform 3.

Preferably, the platform 3 and/or receiving portion 5 are made of a material which is rigid enough to allow the user to push the therapeutic composition C inside the receiving portion 5, and flexible enough to allow the user to push the therapeutic composition C out of the receiving portion 5. The platform 3 and the receiving portion 5 is preferably not so rigid as to injure the patient during the insertion and/or removal process. Preferably, the platform 3 and/or receiving portion 5 are made from a semi-flexible sheet of plastics material.

Where the therapeutic composition is in a fluid, liquid, granular or powder form, the shape and dimensions of the receiving portion 5 may be adjusted to receive the required amount/dosage of therapeutic composition C.

With reference to FIGS. 2A to 2D, the receiving portion 5 may be cup- or bowl-shaped so as to receive a therapeutic composition in a solid or fluid delivery form. A removal string 6 may be connected to the platform 3 so as to pull the device 1 out of the cavity after delivery of the therapeutic composition C.

With reference to FIGS. 3A to 3D and 5, the receiving portion 5 may comprise a flat base 7 (e.g. a cylindrical receiving portion 5) to provide a surface to be pushed by the user.

With reference to FIG. 4, the receiving portion 5 may be a recess or reservoir 8. With reference to FIG. 6, the receiving portion 5 may comprise a visual indication of the amount of therapeutic composition C contained therein.

The receiving portion 5 may be reversible (inside out) to free the therapeutic composition C from the receiving portion 5 (as shown in FIGS. 3A to 3D) or the walls of the receiving portion 5 may be collapsible as shown in FIGS. 7A to 7D. For example, the walls of the receiving portion 5 may comprise a remotely collapsible concertina mechanism 9.

A string or cord 6 may be attached to the platform 3 so as to pull the device 1 out of the cavity after use. The string 6 may also be used to collapse the walls of the receiving portion 5. The string 6 may be attached at a single point to the platform 3 or there may be a plurality of attachment points to facilitate manipulation of the device 1 and/or of the receiving portion 5.

The therapeutic composition C can be in a single solid delivery form, for example a tablet, capsule, pessary, suppository or the like. This solid form will have a specific shape and specific dimensions. With reference to FIGS. 8 to 10, the shape of the receiving portion 5 has a shape which corresponds partially to the shape of the delivery form of the therapeutic composition C.

The sheath 4 is substantially cylindrical and extends from the non-absorbent platform 3 to the pad 2. Preferably, the sheath 4 is made of or comprises a sheet of polymeric biocompatible material. The sheath 4 is arranged and configured to receive a finger. A wide sheath 4 could be provided, however, if the sheath 4 is loose around the user's finger, then insertion becomes difficult. The sheath 4 is preferably extensible at least in the radial direction. An extensible sheath 4 may be provided to snuggly and comfortably fit the user's finger.

The length of the sheath 4 between the pad 2 (which remains outside the cavity) and the platform 3 may be adjusted depending on the therapeutic composition and/or the target area where the drug is to be delivered.

The pad 2 is joined to the sheath 4, more particularly at the second end of the sheath 4. The pad 2 comprises an aperture 10 to allow the user to insert his/her finger into the sheath 4. The pad 2 is substantially circular, oval, rectangular or any other suitable shape. The pad 2 can be a standard rectangular napkin or a small interlabial pad. In a preferred embodiment, the pad 2 is a flat-egg shaped.

The pad 2 comprises a first surface 2A comprising a layer or sheet of absorbent material. The first surface 2A faces, in use, the patient's cavity into which the therapeutic composition C is delivered. The pad 2 comprises a second surface 2B comprising a layer or sheet of impermeable material. The second surface 2B faces way, in use, the patient's cavity. The impermeable material of the pad 2 may be the same material as that of the sheath 4. The sheath 4 may extend through the aperture 10 and along the absorbent layer 2A so as to form the impermeable layer 2B.

The use of the device according to the present invention will be explained with reference to the figures, in particular FIGS. 1, 2, 3, 7, 11 and 12.

In use, the device 1 is positioned so that the absorbent surface 2A of the pad 2 is facing upwards and the impermeable surface 2B is facing downwards. The sheath 4 extends from the impermeable surface 2B of the pad 2 so that the platform 3 and its receiving portion 5 are located at the bottom thereof. Alternatively, the device 1 is position so that the absorbent surface 2A of the pad 2 is facing upwards and the impermeable surface 2B is facing downwards. The sheath 4 extends from the absorbent surface 2A of the pad 2 so that the platform 3 and its receiving portion 5 are located at the top thereof. In both starting configurations, the receiving portion 5 is in its delivery configuration, arranged to receive a therapeutic composition C therein.

The shape of the receiving portion 5 of the device 1 is selected based on the delivery form and shape of the therapeutic composition C. Where the composition C is in a delivery form with a specific shape, the shape and dimensions of the receiving portion 5 will correspond at least partially to the shape and dimensions of the delivery form, for example cylindrical in the case of a suppository. The receiving portion 5 is preferably made of an extensible material so as to secure the therapeutic composition C therein. Where the composition C is of indefinite shape, for example granules, creams, lotions, dispersions, gel and the like, the receiving portion 5 forms a receptacle, for example a cup- or bowl-shaped receptacle to receive the composition C therein. The receiving portion 5 is preferably made of a sufficiently rigid material so as to maintain its shape whilst containing the composition and pushing the platform 3 through the vaginal or rectal cavity. The receiving volume of the receiving portion 5 may be adjusted to contain the required amount of composition C for one application. The sheath 4 or receiving portion 5 may comprise a visual indication of the amount of composition C within the receiving portion 5, for example one or more lines or a graduated line.

The therapeutic composition C is positioned and secured within the receiving portion 5 in the sheath 4. The user inserts his/her finger inside the sheath 4 so that the sheath 4 extends from the absorbent side 2A of the pad 2. The user pushes the platform 3 so that the device 1 is inserted through the cavity orifice. The platform 3 is advanced along through the cavity until it reaches the target area where the composition C is to be delivered and applied. The length of the sheath 4 may be adjusted so that when the sheath 4 is fully extended, the platform 3 is positioned at the target area in the cavity. Because the therapeutic composition C is nested in the receiving portion 5, the user can easily and accurately guide the composition through the cavity.

At the target area, the therapeutic composition is released from the receiving portion 5. This may be effected for example by reversing the receiving portion 5 inside out so that the therapeutic composition is expelled from the receiving portion 5. Alternatively, the receiving portion 5 (or its walls) may be collapsed by pushing the platform 3 to the distal end of the sheath 4 or by using alternative means of collapsing the receiving portion 5 (or its walls). For example, one or more strings 6 may be coupled to the receiving portion 3 so as to remotely collapse the walls of the receiving portion 3 (e.g. by pulling the string 6 from outside the cavity).

In the case where the device 1 comprises a therapeutic composition C pre-positioned in a sealed receiving portion 5, the user inserts his or her finger in the sheath 4 and the sheathed finger into the cavity until the distal end of the sheath 4 reaches the target area. Pressure is applied with the finger onto the sealed composition C to break the seal, reverse the receiving portion, and release the therapeutic composition C.

Once delivered, the therapeutic composition C may be applied to the target area. The present invention is particularly advantageous in that the composition can be rubbed against the cavity walls to improve application to and absorption of the drug through the cavity walls. Tis can be done without the user soiling his/her finger.

The device 1 may be left in situ, so that the therapeutic composition C remains at the target area. The platform 3 act as an anchor and/or plug for example until the composition has dissolved and been absorbed.

Any excess fluid leaking from the cavity is absorbed by the absorbent surface 2A of the pad 2 which is located at the cavity orifice. The user's finger is protected from said fluids owing to the impermeable surface 2B of the pad 2.

The device 1 is removed either by pulling the pad 2 or the string 6. By pulling the string 6, the sheath 4 is reversed so that the soiled outer surface of the sheath 4 is now inside. The user can hold the device 1 by the unsoiled surface of the sheath 4 or the impermeable surface 2B of the pad 2, and dispose of the device 1.

The present invention is particularly useful in the delivery and application of therapeutic composition to relieve the symptoms of menorrhagia. Menorrhagia is a condition wherein women suffer from excessive bleeding at menstruation. This may be through heavy menstrual bleeding (80 ml or more) and/or prolonged menstrual periods (7 days or more). The causes of menorrhagia vary from hormonal imbalance, the use of an intrauterine birth control device to complications in pregnancy. Many treatments are available to alleviate the symptoms of menorrhagia, including:
Non-steroidal and/or non/hormonal drugs, for example NSAIDs and painkillers can reduce bleeding;
Hormonal drugs, for example contraceptive pills, or hormone coils for contraception, but some patients with menorrhagia might still want children;
Surgery, where non-invasive treatments fail, for example endometrial ablation

For these patients who do not wish to use a contraceptive treatment or refuse surgery, the present invention offers a non-invasive treatment involving the delivery of non-hormonal drugs (for example pain killers) to treat the symptoms in situ. The present invention is particularly advantageous in that the treatment will be carried out during periods of heavy bleeding, when the application of medicine in the vaginal cavity will be particularly difficult and messy because of the abundance of bodily fluids. When the drug is delivered and applied, the sheath protects the patient's finger and enables the application and rubbing of the drug against the cavity walls. The external pad soaks in any bodily fluid exiting the cavity during the delivery and application of the drug.

The present invention is particularly useful in the context of fertility treatments, in particular treatments such as In-Vitro Fertilization (IVF), involving the administration of various drugs such as hormones and anti-coagulants, often as pessaries, tablets or injected (sub-cutaneous or intramuscular). The vast majority of IVF medications are self-administered and there is a need for simpler, more patient-friendly administration method, without compromising the delivery of the therapeutic composition.

During an IVF treatment, women will be required to take progesterone around three times a day. Progesterone cannot be taken orally because of bioavailability issues. In order to efficiently deliver the required amount of progesterone to the womb, the ingested amount would need to be high enough to cause undesirable side-effects. Another option is to inject progesterone in situ. However, it would be impractical, painful and expensive to inject progesterone into the vagina. The most common administration routes are vaginal suppositories inserted with a finger or using an applicator. In order to be effective, the suppository must remain in place to dissolve and be absorbed. However, in practice, the majority of the progesterone will leak out and it is therefore not possible to administer an accurate amount. It is also not possible to wear a tampon to prevent leakage, otherwise the progesterone will be absorbed by the tampon.

The present invention enables is an alternative to conventional rigid applicators and enables the comfortable and clean delivery and application of the suppository. The suppository can be maintained at the target area until it is fully dissolved and, since the finger is protected, the dissolved composition can be rubbed against the vaginal cavity to optimize drug delivery.

The present invention provides a device for the delivery and/or application of a therapeutic composition into the rectal or vaginal cavity. From the above description, it can be seen that the device is used as a drug inserter which enables the pain-free and hygienic delivery and application of an accurate amount of therapeutic composition to a target area.

1. A device for the delivery and/or application of a therapeutic composition in a delivery form into the rectal and/or vaginal cavity, the device comprising:
an externally wearable pad;
a non-absorbent platform for receiving the delivery form of therapeutic composition; and
a sheath joining the platform to the pad such that a user's finger can be received in the sheath.

2. The device according to claim 1, wherein the therapeutic composition is a delivery form with a shape, and the platform comprises a receiving portion, the shape of which corresponds partially to the shape of the delivery form of the therapeutic composition.

3. The device according to claim 1, wherein the platform comprises a receiving portion which is adapted and configured to receive a predetermined amount of therapeutic composition.

4. The device according to claim 1, wherein the platform comprises an area of relatively greater rigidity.

5. The device according to claim 2, wherein the receiving portion is collapsible and/or reversible.

6. The device according to claim 1, wherein the platform comprises means for releasing the therapeutic composition.

7. The device according to claim 1, further comprising a removal string.

8. The device according to claim 1, wherein the pad comprises an absorbent layer facing, in use, the rectal or vaginal cavity and an impermeable layer facing, in use, away from the rectal or vaginal cavity.

9. The device according to claim 1, wherein the length of the sheath is substantially the same as the distance between the rectal or vaginal orifice a target area in the rectal or vaginal cavity.

10. A kit comprising a therapeutic composition in a delivery form and a device according to claim 1 for delivering and/or applying said delivery form of the therapeutic composition to a target area in the rectal or vaginal cavity.

11. The kit according to claim 10, wherein the therapeutic composition comprises an anti-fungal agent, an anti-microbial agent, an anti-inflammatory agent, a pain killer, a steroidal (NSAID) compound, a non-steroidal compound, and/or a hormone.

12. The use of the device according to claim 1, for the delivery and/or application of a therapeutic composition for the treatment of menorrhagia.

13. The use of the device according to claim 1, for the delivery and/or application of a therapeutic composition for fertility treatment.
